(19) United States
(12) Patent Application Publication
(10) Pub. No.: US 2005/0227911 A1
(43) Pub. Date: Oct. 13, 2005

(54) Hydrophilic dispersions of nanoparticles of inclusion complexes of macromolecules

(75) Inventors: Rina Goldshtein, Har Hebron (IL); Galina Ratner, Rehovot (IL); Larisa Gitis, Holon (IL); Boris Tulbovich, Ashkelon (IL)

Correspondence Address: BROWDY AND NEIMARK, P.L.L.C.;624 NINTH STREET, NW, SUITE 300, WASHINGTON, DC, 20001-5303, US

(73) Assignee: SoluBest Ltd., Rehovot (IL)

(21) Appl. No.: 11/100,622

(22) Filed: Apr. 7, 2005

Related U.S. Application Data
(63) Continuation-in-part of application No. 10/952,380, filed on Sep. 29, 2004.
(63) Continuation-in-part of application No. 10/256,023, filed on Sep. 26, 2002.
Application No. 10/256,023 is a continuation-in-part of application No. 09/966,847, filed on Sep. 28, 2001, now Pat. No. 6,878,693.
(60) Provisional application No. 60/507,623, filed on Sep. 30, 2003.

(51) Int. Cl. A61K 38/00; C07H 21/04; A61K 48/00; C07K 14/47

(52) U.S. Cl. 514/2; 514/60; 514/44; 530/395; 536/23.1

(57) ABSTRACT

The present invention provides hydrophilic dispersions comprising nanoparticles of inclusion complexes consisting essentially of nanosized particles of a macromolecule wrapped in an amphiphilic polymer such that non-valent bonds are formed between the macromolecule and the amphiphilic polymer. The macromolecules may be a naturally-occurring, synthetic or recombinant polypeptide, protein, polysaccharide or polynucleotide, and the amphiphilic polymer is a polysaccharide or a modified polysaccharide such as starch, chitosan or an alginate.

### CROSS-REFERENCE TO RELATED APPLICATIONS

The present application is a continuation-in-part of application Ser. No. 10/952,380, filed Sep. 29, 2004, which is a non-provisional of the Provisional Application No. 60/507,623, filed Sep. 30, 2003 and a continuation-in-part of application Ser. No. 10/256,023, filed Sep. 26, 2002, which is a continuation-in-part of application Ser. No. 09/966,847, filed Sep. 28, 2001 each and all these applications being hereby incorporated by reference herein in their entirety as if fully disclosed herein.

### FIELD OF THE INVENTION

The present invention is in the field of nanoparticles. More particularly, the invention relates to soluble nanosized particles consisting of inclusion complexes of an active macromolecule wrapped within a suitable amphiphilic polymer, and methods of producing said nanoparticles.

### BACKGROUND OF THE INVENTION

Two formidable barriers to effective drug delivery and hence to disease treatment, are solubility and stability. To be absorbed in the human body, a compound has to be soluble in both water and fats (lipids). Solubility in water is, however, often associated with poor fat solubility and vice-versa.

Over one third of drugs listed in the U.S. Pharmacopoeia and about 50% of new chemical entities (NCEs) are insoluble or poorly insoluble in water. Over 40% of drug molecules and drug compounds are insoluble in the human body. In spite of this, lipophilic drug substances having low water solubility are a growing drug class having increasing applicability in a variety of therapeutic areas and for a variety of pathologies.

Solubility and stability issues are major formulation obstacles hindering the development of therapeutic agents. Aqueous solubility is a necessary but frequently elusive property for formulations of the complex organic structures found in pharmaceuticals. Traditional formulation systems for very insoluble drugs have involved a combination of organic solvents, surfactants and extreme pH conditions. These formulations are often irritating to the patient and may cause adverse reactions.

The size of the drug molecules also plays a major role in their solubility and stability as well as bioavailability. Bioavailability refers to the degree to which a drug becomes available to the target tissue or any alternative in vivo target (ie., receptors, tumors, etc.) after being administered to the body. Poor bioavailability is a significant problem encountered in the development of pharmaceutical compositions, particularly those containing an active ingredient that is poorly soluble in water. Poorly water-soluble drugs tend to be eliminated from the gastrointestinal tract before being absorbed into the circulation. It is known that the rate of dissolution of a particulate drug can increase with increasing surface area, that is, decreasing particle size

Recently, there has been an explosion of interest in nanotechnology, the manipulation on the nanoscale. Nanotechnology is not an entirely new field: colloidal sols and supported platinum catalysts are nanoparticles. Nevertheless, the recent interest in the nanoscale has produced, among numerous other things, materials used for and in drug delivery. Nanoparticles are generally considered to be solids whose diameter varies between 1-1000 nm.

Although a number of solubilization technologies do exist, such as liposomes, cylcodextrins, microencapuslation, and dendrimers, each of these technologies has a number of significant disadvantages.

Liposomes, as drug carriers, have several potential advantages, including the ability to carry a significant amount of drug, relative ease of preparation, and low toxicity if natural lipids are used. However, common problems encountered with liposomes include: low stability, short shelf-life, poor tissue specificity, and toxicity with non-native lipids. Additionally, the uptake by phagocytic cells reduces circulation times. Furthermore, preparing liposome formulations that exhibit narrow size distribution has been a formidable challenge under demanding conditions, as well as a costly one. Also, membrane clogging often results during the production of larger volumes required for pharmaceutical production of a particular drug.

Cyclodextrins are crystalline, water-soluble, cyclic, non-reducing oligo-saccharides built from six, seven, or eight glucopyranose units, referred to as alpha, beta and gamma cyclodextrin, respectively, which have long been known as products that are capable of forming inclusion complexes. The cyclodextrin structure provides a molecule shaped like a segment of a hollow cone with an exterior hydrophilic surface and interior hydrophobic cavity. The hydrophilic surface generates good water solubility for the cyclodextrin and the hydrophobic cavity provides a favorable environment in which to enclose, envelope or entrap the drug molecule. This association isolates the drug from the aqueous solvent and may increase the drug's water solubility and stability.

For a long time, most cyclodextrins had been no more than scientific curiosities due to their limited availability and high price, but lately cyclodextrins and their chemically modified derivatives became available commercially, generating a new technology of packing on the molecular level. Cyclodextrins are, however, fraught with disadvantages including limited space available for the active molecule to be entrapped inside the core, lack of pure stability of the complex, limited availability in the marketplace, and high price.

Microencapsulation is a process by which tiny parcels of a gas, liquid, or solid active ingredient (“core material”) are packaged within a second material for the purpose of shielding the active ingredient from the surrounding environment. These capsules, which range in size from one micron (one-thousandth of a millimeter) to approximately seven millimeters, release their contents at a later time by means appropriate to the application.

There are four typical mechanisms by which the core material is released from a microcapsule: (1) mechanical rupture of the capsule wall, (2) dissolution of the wall, (3) melting of the wall, and (4) diffusion through the wall. Less common release mechanisms include ablation (slow erosion of the shell) and biodegradation.

Microencapsulation covers several technologies, where a certain material is coated to obtain a micro-package of the active compound. The coating is performed to stabilize the material, for taste masking, preparing free flowing material of otherwise clogging agents etc. and many other purposes. This technology has been successfully applied in the feed additive industry and to agriculture. The relatively high production cost needed for many of the formulations is, however, a significant disadvantage.

In the cases of nanoencapsulation and nanoparticles (which are advantageously shaped as spheres and, hence, nanospheres), two types of systems having different inner structures are possible: (i) a matrix-type system composed of an entanglement of oligomer or polymer units, defined as nanoparticles or nanospheres, and (ii) a reservoir-type system, consisting of an oily core surrounded by a polymer wall, defined as a nanocapsule.

Depending upon the nature of the materials used to prepare the nanospheres, the following classification exists: (a) amphiphilic macromolecules that undergo a cross-linking reaction during preparation of the nanospheres; (b) monomers that polymerize during preparation of the nanoparticles; and (c) hydrophobic polymers, which are initially dissolved in organic solvents and then precipitated under controlled conditions to produce nanoparticles.

Problems associated with the use of polymers in micro- and nanoencapsulation include the use of toxic emulgators in emulsions or dispersions, polymerization or the application of high shear forces during emulsification process, insufficient biocompatibility and biodegradability, balance of hydrophilic and hydrophobic moieties, etc. These characteristics lead to insufficient drug release.

Dendrimers are a class of polymers distinguished by their highly branched, tree-like structures. They are synthesized in an iterative fashion from ABn monomers, with each iteration adding a layer ort “generation” to the growing polymer. Dendrimers of up to ten generations have been synthesized with molecular weights in excess of 106 kDa. One important feature of dendrimeric. polymers is their narrow molecular weight distributions. Indeed, depending on the synthetic strategy used, dendrimers with molecular weights in excess of 20 kDa can be made as single compounds.

Dendrimers, like liposomes, display the property of encapsulation, and are able to sequester molecules within the interior spaces. Because they are single molecules, not assemblies, drug-dendrimer complexes are expected to be significantly more stable than liposomal drugs. Dendrimers are thus considered as one of the most promising vehicles for drug delivery systems. However, the dendrimer technology is still in the research stage, and it is speculated that it will take years before it is applied in the industry as an efficient drug delivery system.

Efficient delivery of macromolecular drugs is a very timely issue today. Biopharmaceuticals continue to play a greater and greater role in the pharmaceutical industry and it is anticipated that this trend will increase significantly. There are several major limitations currently on using large biomolecules in the pharmaceutical field; this includes their poor aqueous solubility and their relative instability in in vivo administration, in particular via the oral route. Concurrently, macromolecules present an extraordinary challenge with regard to their formulation, being generally labile molecules with difficulties in passing biological barriers.

There is a need to provide preparations of biomacromolecules such as peptides, proteins, nucleic acids and carbohydrates used therapeutically or otherwise, that are biocompatible and stable and efficiently deliver the biomacromolecule for therapeutical or other use.

### SUMMARY OF THE INVENTION

It has now been found by the present inventors that the technology of solumerization disclosed in the above-mentioned parent U.S. application Ser. Nos. 10/952,380 and 10/256,023, incorporated herewith by reference in their entirety, for small organic compounds, can be applied also for macromolecules.

The present invention thus relates to a hydrophilic inclusion complex consisting essentially of nanosized particles of a macromolecule wrapped in an amphiphilic polymer such that non-valent bonds are formed between the macromolecule and the amphiphilic polymer, wherein said amphiphilic polymer is a polysaccharide or a modified polysaccharide.

The present invention further relates to hydrophilic dispersions comprising nanoparticles of said inclusion complexes, to their preparation and to stable pharmaceutical compositions comprising said dispersions.

### BRIEF DESCRIPTION OF THE DRAWINGS

FIG. 1 is a graph showing the stability of the inclusion complex of 0.1% sodium hyaluronate (NaHA) with 1.9% hydrolyzed potato starch (HPS) or with 1.9% modified corn starch B-790 (B-790) in the presence of hyaluronidase, measured after 3 and 5 hours. Viscosity (% based on initial).

FIG. 2 is a graph showing the stability of the inclusion complex of 0.5% sodium hyaluronate (NAHA) with 1.9% hydrolyzed potato starch (HPS) or with 1.9% modified corn starch B-790 (B-790) in the presence of hyaluronidase, measured after 3 and 5 hours. Viscosity (% based on initial).

### DETAILED DESCRIPTION OF THE INVENTION

The present invention provides nanoparticles and methods for the production of soluble nanoparticles and, in particular, hydrophilic dispersions of nanoparticles of inclusion complexes of an active macromolecular compound in certain amphiphilic polymers.

The soluble nanoparticles, referred to herein sometimes as “solu-nanoparticles” or “solumers”, are differentiated by the use of water-soluble amphiphilic polymers that are capable of producing molecular complexes with active macromolecules, particularly pharmaceutical drugs. The solunanoparticles formed in accordance with the present invention render water-insoluble active compounds soluble in water and readily bioavailable in the human body.

As used herein, the term “inclusion complex” refers to a complex in which one component—the amphiphilic polymer (the “host”), forms a cavity in which molecular entities of a second chemical species—the active compound (the “guest”), are located. Thus, in accordance with the present invention, inclusion complexes are provided in which the host is the amphiphilic polymer and the guest is the active macromolecule wrapped and fixated or secured within the cavity or space formed by said amphiphilic polymer host.

In accordance with the present invention, the inclusion complexes contain the active macromolecules, which interact with the polymer by non-valent interactions and form a polymer-active macromolecular compound as a distinct molecular entity. A significant advantage and unique feature of the inclusion complex of the present invention is that no new chemical bonds are formed and no existing bonds are destroyed during the formation of the inclusion complex (very important for pharmaceutical drugs). The particles comprising the inclusion complexes are nanosized and no change occurs in the active macromolecular compound molecule itself, when it is enveloped, or advantageously wrapped, by the polymer.

Another important characteristic of the inclusion complex of the invention is that the active macromolecular compound may be presented in a non-crystalline state. As used herein, the term “non-crystalline” state is intended to include both disordered crystalline and, preferably, amorphous state. Thus, in preferred embodiments, the active compound is in amorphous form. It is known in the art that the amorphous state is preferred for drug delivery as it may indeed enhance bioavailability.

The creation of the complex does not involve the formation of any valent bonds (which may change the characteristics or properties of the active macromolecular compound). As used herein, the term “non-valent” is intended to refer to non-covalent, non-ionic and non-semi-polar bonds and/or interactions, and includes weak, non-covalent bonds and/or interactions such as electrostatic forces, Van der Waals forces, and hydrogen bonds formed during the creation of the inclusion complex. The formation of non-valent bonds preserves the structure and roperties of the active macromolecular compound.

The solunanoparticles of the invention remain stable for long periods of time, may be manufactured at a low cost, and may improve the overall bioavailability of the active compound.

In one aspect, the present invention relates to a hydrophilic inclusion inclusion complex consisting essentially of nanosized particles of a macromolecule enveloped or wrapped in certain amphiphilic polymers.

As defined herein, a “macromolecule” is a naturally-occurring, recombinant or synthetic macromolecule of large molecular weight, usually above 1,000 Da, exhibiting biological activity, and includes polypeptides, proteins, nucleic acids and polysaccharides.

In one embodiment, the macromolecule is a naturally-occurring, recombinant or synthetic polypeptide of molecular weight above 1,000 Da or protein such as hormones, enzymes, immunoglobulins, monoclonal antibodies, cytokines or chemokines, and the like. Some of these proteins are used as pharmaceuticals or are in clinical trials or have the potential to be used as pharmaceuticals.

In one embodiment, the protein is a hormone such as insulin, human growth hormone, luteinizing hormone (LH) and human chorionic gonadotropin (hCG).

In another embodiment, the protein is a cytokine or chemokine such as, but not limited to, an interferon (IFN) such as IFN-α, IFN-β, IFN-γ, an interleukin (IL) such as any of the known IL-1 to IL-18, or a member of the IL-6 family such as LIF, OSN and CNTF, a chemokine, a hematopoietic colony-stimulating factor (CSF) such as granulocyte colony-stimulating factor (G-CSF), granulocyte-macrophage colony-stimulating factor (GM-CSF), and macrophage colony-stimulating factor (M-CSF), a tumor necrosis factor (TNF) such as TNF-α and TNF-β also called lymphotoxin, or a member of the TNF superfamily such as NGF and FAS-Ligand (FASL), a transforming growth factor (TGF) such as TGF-α and TGF-β, erythropoietin (EPO) and thymopoietin (TPO).

In a further embodiment, the protein is an enzyme, in particular, enzymes used in therapy such as, but not limited to, trypsin, chymotrypsin, pancreatin, papain, bromelain, fibrinolysin, streptokinase, tissue plasminogen activator (TPA or tPA), urokinase, hyaluronidase (an enzyme that catalyzes the breakdown of hyaluronic acid in the body, thereby increasing tissue permeability to fluids, also called ‘spreading factor’), acid beta-glucocerebrosidase (used in enzyme replacement therapy for patients with Gaucher disease), peroxidases, and superoxide dismutase (SOD).

In another embodiment, the macromolecule is a polysaccharide such as, but not limited to, lentinan and a member of the glycosaminoglycan (GAG) family such as hyaluronic acid (HA) and its salts,. particularly sodium hyaluronate, chondroitin sulphate, dermatan sulphate, heparan sulphate, and heparin and its derivatives including low molecular weight heparins (LMWH).

Hyaluronic acid (HA) is a naturally occurring biopolymer comprised of linear, unbranching, polyanionic disaccharide units consisting of glucuronic acid (GlcUA) an N-acetyl glucosamine (GlcNAc) joined alternately by beta 1-3 and beta 1-4 glycosidic bonds. Unlike other members of the glycosaminoglycan family, it is not found covalently bound to proteins. HA is a significant component of a number of bodily tissues and fluids, e.g., the extracellular matrix of cartilage, synovial fluid, and the vitreous humor of the eye. HA can be isolated from mammalian and avian tissues or certain strains of cultured bacteria, as is known in the art.

Commercial HA is commonly its sodium salt form (NaHA) and both are available for several pharmaceutical and cosmetic uses. HA moisturizes skin, reduces wrinkles and acts to cushion the joints and can be used as anti-aging agent in cosmetic products as well as in pharmaceutical products, for example, as injections in the treatment of osteoarthritic knees as a joint lubrication fluid. Sodium hyaluronate has been known as moisturizer since its solution is highly viscous and is also indicated for the treatment of pain in osteoarthritis of the knee in patients who have failed to respond adequately to conservative non-pharmacologic therapy and simple analgesics (e.g., acetaminophen).

The nanoparticles of the present invention comprise the active macromolecular compound or core wrapped within a water-soluble amphiphilic polymer. As described in the parent U.S. application Ser. Nos. 10/256,023 and 09/966,847, hereby incorporated by reference in their entirety, a variety of different polymers can be used for any of the selected active compounds. The polymer used in the formation of the nano-soluparticles are selected according to an algorithm that takes into account various physical properties of the active compounds and the polymer or polymers, as well as their future interaction in the resulting complex. The algorithm is utilized in this manner to select the optimal polymer(s) and takes into consideration the following properties of the polymer itself in selecting a polymer for the active molecule/polymer interaction in the formation of the complex: molecular weight, basic polymer chain length, the length of the kinetic unit, the solubility of the polymer in water, the overall degree of solubility, the degree of polymer flexibility, the hydrophilic-lipophilic balance (HLB), and the polarity of the hydrophilic groups of the polymer. The main properties of the polymer include its HLB, the length and the flexibility of its polymer chain, and also the state of polarity of the hydrophilic groups.

Thus, one important parameter in the choice of the polymer or polymers is the HLB, i.e., the measure of the molecular balance of the hydrophilic and lipophilic portions of the compound. Within the HLB International Scale of 0-20, lipophilic molecules have a HLB of less than 6, and hydrophilic molecules have a HLB of more than 6. Thus, according to the present invention, the HLB of the polymer is selected in such a way that, after combining to it the active compound, the total resulting HLB value of the complex will be greater than 8, rendering the complex water-soluble.

In accordance with the present invention, the amphiphilic polymers found suitable for the preparation of inclusion complexes with the macromolecules are polysaccharides, in natural form or modified. The polysaccharide may be starch, chitosan or an alginate.

In one embodiment, the polysaccharide is starch. The starch should preferably have a large proportion of linear chains, i.e. starch with high contents of amylose, the constituent of starch in which anhydroglucose units are linked by (-D)-1,4 glucosidic bonds to form linear chains, and low contents of amylopectin,a constituent of starch having a polymeric, branched structure. The levels of amylose and amylopectin and their molecular weight vary between different starch types. Encompassed by the present invention are starches of various sources such as potato, maize/corn, wheat, and tapioca/cassava starch.

To improve its characteristics for use in the invention, starch, e.g. corn or potato starch, can be modified, for example by increasing its hydrophilicity by acid hydrolysis, e.g., with citric acid, and/or by reaction with an agent, e.g. polyethylene glycol (PEG) and/or hydrogen peroxide. In addition, starch can be subjected to thermal and pressure treatment to reduce the amount of branching (designated “thermodestructed starch”). Some modified starch products are commercially available and can be used according to the invention.

Thus, in one embodiment, the nanoparticles of the invention consist of inclusion complexes in which the active compound is a macromolecule and the amphiphilic polysaccharide is modified starch selected from the group consisting of hydrolyzed starch, starch modified by different amounts of PEG, preferably PEG-400, and/or by H₂O₂, pregelatinized starch and thermodestructed starch.

For the preparation of pregelatinized starch, the aqueous mixture of starch is heated, for example, at 70-80° C., for about 180-190 min. For thermal modification, the aqueous mixture of starch can be autoclaved for about 50-60 minutes (110-115° C. and pressure 1.5-1.6 atm). Under these conditions, the network structures of starch are partially or completely transformed to linear weakly branched macromolecules which dissolve better in water.

In one embodiment, the present invention relates to an inclusion complex of hyaluronic acid wrapped in modified starch, wherein the modified starch is hydrolyzed potato starch modified by reaction with hydrogen peroxide or the modified corn starch B-790 (Pure-Cote® B-790, Grain Processing Corp., Muscatine, Iowa, USA).

In another embodiment, the present invention relates to an inclusion complex of bovine serum albumin wrapped in the modified corn starch B-790.

In one further embodiment, the present invention relates to an inclusion complex of hyaluronidase wrapped in the modified corn starch B-790.

In another aspect, the present invention provides a hydrophilic dispersion comprising nanoparticles of inclusion complexes as defined above. Thus, the present invention provides a hydrophilic dispersion of water-soluble and stable nanoparticles of inclusion complexes consisting essentially of nanosized particles of an active macromolecule and an amphiphilic polymer consisting of a polysaccharide or modified polysaccharide which wraps said active macromolecule such that non-valent bonds are formed between said active macromolecule and said amphiphilic polymer in said inclusion complex.

The dispersions of the invention are stable. Stability of the nanoparticles and of the inclusion complexes has more than one meaning. The nanoparticles should be stable as part of a nanocomplex over time, while remaining in the dispersion media. The nanodispersions are stable over time without separation of phases. Furthermore, the non-crystalline or amorphous state should be also retained over time.

It is worth noting that in the process used in the present invention, the components of the system do not result in micelles nor do they form classical dispersion systems. The technology of the present invention causes the following:

(i) after dispersion of the active macromolecule to nanosized particles and fixation by the polymer to form an inclusion complex, enhanced solubility in physiological fluids, in vivo improved absorption, and improved biological activity, as well as transmission to a stable non-crystalline, preferably amorphous, state, are achieved; and

(ii) the otherwise crystalline biologically-active macromolecule becomes non-crystalline, e.g., amorphous, and thus exhibits improved biological activity.

In most preferred embodiments of the present invention, not less than 80% of the nanoparticles in the nanodispersion are within the size range, when the size deviation is not greater than 20%, and the particle size is within the nano range, namely less than 1000 nm, more preferably 100 nm or less.

In an advantageous and preferred embodiment of the invention, the polysaccharide molecule “wraps” the active macromolecule via non-valent interactions. The non-valent bonds or interactions such as electrostatic forces, Van der Waals forces, and hydrogen-bonds formed between the polysaccharide and the active macromolecule in the inclusion complex fixate the active macromolecule within the polysaccharide, thus reducing its molecular mobility. The formation of any valent bonds could change the characteristics or properties of the active macromolecule. The formation of non-valent bonds preserves the structure and properties of the active macromolecule, which is particularly important when the active macromolecule is a pharmaceutical.

The technology developed according to the present invention for macromolecules has the ability to be applied in a very versatile manner to a broad spectrum of active macromolecular compounds with vastly different molecular weights ranging from 1,000 and up to millions Da, stemming from different types of macromolecules having different functionalities and different 3D structures. It is demonstrated herein that this technology has the ability to create nanosized transport systems with several different macromolecules and in so has shown them to have greater stability in the presence of acids and specific enzymatic systems. Nanosize obtainment not only succeeds in compressing these macromolecules into nanotransport systems but as such gives them significantly increased surface area and volume, which as anticipated will allow them to increase their bioavailability and efficacy while attaining increased stability.

Contrary to the process for the preparation of the hydrophilic dispersions comprising the nanoparticles of the inclusion complexes of non-macromolecular compounds disclosed in the parent U.S. application Ser. No. 10/952,380 and 10/256,023, hereby incorporated by reference in their entirety, whereby the polymer is first dissolved in an aqueous solution and a molecular solution of the active compound in an organic solvent is added to the polymer aqueous solution, it was found according to the present invention that, when the active compound is a macromolecule, the reaction can be carried out without an organic solvent.

The present invention thus provides a process for preparation of a hydrophilic dispersion comprising nanoparticles of inclusion complexes of an active macromolecule and an amphiphilic polysaccharide which wraps the active macromolecule such that non-valent bonds are formed between said active macromolecule and said amphiphilic polysaccharide, the process comprising the steps of:

(i) preparing a solution of the amphiphilic polysaccharide in water;

(ii) preparing a molecular solution of the active macromolecule in water and

(iii) adding dropwise the water solution of the active macromolecule (ii) into the water polysaccharide solution (i) under constant mixing;

thus obtaining the hydrophilic dispersion comprising nanoparticles of inclusion complexes of said active macromolecule wrapped within said amphiphilic polysaccharide.

In step (ii), the macromolecule aqueous solution is treated with a salt, for example, ammonium sulfate, KCl or NaCl, before addition to the polymer water solution. In step (iii), the macromolecule is added to the warmed polysaccharide solution, when the macromolecule is not a protein, as shown herein for hyaluronic acid. When the macromolecule is a protein, as shown herein for bovine serum albumin and hyaluronidase, the macromolecule is added to the polysaccharide solution at room temperature.

The aqueous nanodispersions of the invention can be lyophilized and then mixed with pharmaceutically acceptable carriers to provide stable pharmaceutical compositions.

The pharmaceutically acceptable carriers or excipients are adapted to the active compound and the type of formulation and can be chosen from standard excipients as well-known in the art, for example, as described in Remington: The Science and Practice of Pharmacy (Formerly Remington's Pharmaceutical Sciences) 19th ed., 1995.

Thus, in another aspect, the present invention provides stable pharmaceutical compositions comprising pharmaceutically acceptable carriers and a nano-dispersion of the invention comprising nanoparticles of a macromolecule. The compositions may be in liquid or solid form, and may be administered by any suitable mode of administration including oral and injectable formulations.

In a preferred embodiment, the invention relates to a stable pharmaceutical or cosmetic composition comprising a dispersion of nanoparticles of hyaluronic acid according to the invention and a pharmaceutically acceptable carrier.

In another embodiment, the invention relates to a stable cosmetic composition comprising a dispersion of nanoparticles of hyaluronic acid according to the invention and a cosmetically acceptable carrier.

In a further embodiment, the invention relates to a stable pharmaceutical composition comprising a dispersion of nanoparticles of hyaluronidase according to the invention and a pharmaceutically acceptable carrier.

The invention will now be illustrated by the following non-limiting examples.

### EXAMPLES

### Example 1

### Preparation of Polymers (Modified Starch)

(i) Hydrolyzed potato starch (HPS) 3.8% with H₂O₂ (1%)—Polymer A

Polymer A was prepared by adding 20 g potato starch to 500 ml of water, adding 0.2 ml of 20% citric acid and mixing. Autoclaving was carried out for 60 min (1.58-1.61 atm, 113-115° C.). Hydrogen peroxide was added (15 ml 33% H₂O₂) at temperature 67° C. under mixing with magnet stirrer for 60 minutes. After cooling to room temperature, pH, turbidity and viscosity of the solution were measured. The values obtained were: pH 3.5±0.4, turbidity 33±2 FTU (formazin turbidity unit), and viscosity 20±2 cP (centipoises).

In this and in the following examples, turbidity was measured with a SMART 2 calorimeter (LaMotte Company, Chestertown, Mass., USA), using the turbidity mode for this measurement; viscosity was measured with Visco Star Plus (measurements were made at a room temperature, spindle TL5, 100 rpm).

(ii) Modified Food (Corn) Starch B-790 (Pure-Cote B-790®, Grain Processing Corp., Muscatine, Iowa, USA) 3.8%—Polymer B

A solution of Polymer B was prepared by adding 24 g modified corn starch B-790 to 600 ml of water under mixing with magnetic stirrer and heating at 70-80° C. for 180±10 min. After cooling to room temperature, the mixture was filtered through the filter paper MN 615 ¼, and pH, turbidity and viscosity of the solution were measured. The values obtained were: pH 5.5±0.3, turbidity 200±10 FTU and viscosity 10±2 cP.

### Example 2

### Preparation of Solu-Sodium Hyaluronate (Solu-NaHA), 0.1%

Preparation of 0.2% solutions of sodium hyaluronate of two different molecular weights (NaHA; MW 3 million Da and 1.3 million Da, NaHA from human umbilical cord, Sigma, H 1876) was carried out by dissolution of 0.2 g of NaHA in 100 ml water at room temperature with mixing on magnet stirrer without heating during 120±10 min.

NaCl was added to the final concentration of 1.7% (w/w): 1.7 g NaCl to 100 ml 0.2% solution of NaHA, and mixed for 5-10 min. 50 ml of Polymer A or Polymer B were placed in a three-necked flask of 150 ml and heated in a water-bath up to the temperature 54-56° C. An equal volume (50 ml) of 0.2% NaHA solution was added dropwise to the polymer solution (0.35 ml in 1 minute) with constant mixing by a mechanical glass stirrer utilizing a teflon tip (stirring rate-300 rpm). Upon completion, the solution was cooled under constant mixing at 30-32° C. The final product, herein designated Solu-NaHA, is an opalescent solution, concentration of NaHA-0.1%.

The pH, viscosity and size of the particles were measured. Viscosity was measured by Visco Star Plus (Fungilab SA, Spain) at room temperature; size of particles was measured by dynamic light scattering with a Malvern Zeta Sizer. The values obtained were: pH 4.0±0.5, viscosity 13±2 cP. The average particle diameter of the Solu-NaHA was 100-130 nm.

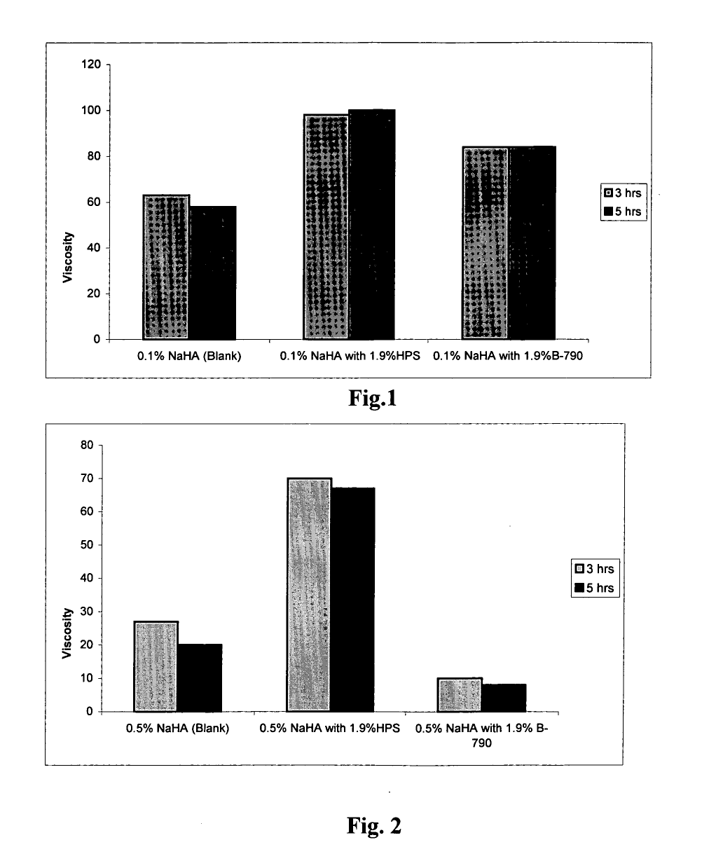

The stability of Solu-NaHA in the presence of the enzyme specific for hyaluronic acid, hyaluronidase (Sigma, H 3506, Hyaluronidase lyophilized (EC 3.2.1.35) Type I-S, from bovine testes, 608 U/mg solid) was measured by the decrease of the viscosity in time in comparison to blank. The degree of stability (protection against action of hyaluronidase,) is defined as a decrease in the viscosity of the NaHA solution upon addition of the enzyme (dose of enzyme−10 U/ml). The control used was 0.1% solution of NaHA without the wrapping polymer (blank). Samples were maintained on a water bath at temperature 37° C. during 5 hrs. Sample made by Visco Star Plus. The decrease in viscosity was estimated in percentage to its initial value. The results in FIG. 1 show the stability of Solu-NaHA prepared with polymer A (NaHA-HPS) or with Polymer B (NaHA-B-790) against the action of hyaluronidase, established as 84-100% vs control 58-63%.

### Example 3

### Preparation of Sodium Hyaluronate (Solu-NaHA), 0.5%

Preparation of 1% solutions of sodium hyaluronate of two different molecular weights [NaHA; MW 3 million Da and 1.3 million Da, SIGMA, H 1876, Hyaluronic acid sodium salt from human umbilical cord] was carried out by dissolution of 10 g of NaHA in 100 ml water at room temperature with mixing on magnet stirrer without heating during 300±30 min.

NaCl was added to the final concentration of 1.7% (w/w): 1.7 g NaCl to 100 ml 1.0% solution of NaHA, and mixed for 5-10 min. 50 ml of Polymer A or Polymer B were placed in a three-necked flask of 150 ml and heated in a water-bath up to the temperature 54-56° C. An equal volume (50 ml) of 1.0% NaHA solution was added dropwise to the polymer solution (0.35 ml in 1 minute) with constant mixing by a mechanical glass stirrer utilizing a teflon tip (stirring rate: 300 rpm). Upon completion, the solution was cooled under constant mixing at 30-32° C. The final product, herein designated Solu-NaHA, is an opalescent solution, concentration of NaHA-0.5%.

The pH, viscosity and size of the particles were measured as described in Example 2. The values obtained were: pH 4.0±0.5, viscosity 50±10 cP. The average particle diameter of the Solu-NaHA was 100-140 nm.

The stability of NaHA in the Solu-NaHA in the presence of hyaluronidase was measured by the decrease of the viscosity over time in comparison to blank. The results are shown in FIG. 2. The stability of Solu-NaHA against the action of hyaluronidase was 70-90% vs 40-45% in blank (0.5% solution of NaHA).

The degree of stability of NaHA was measured as described in Example 2 above. The control used was 0.5% solution of NaHA without any polymer (blank). Samples were maintained on a water bath at temperature 37° C. during 5 hrs. Measurement of viscosity was made by Visco Star Plus. Decrease in viscosity is estimated in percentage relative to its initial value. It is established, that protection of Solu-NaHA with polymer A (HPS) against action of hyaluronidase gives a viscosity of 67-70% vs the control protection of 20-27%. However, Solu-NaHA with polymer B (B-790) does not show such activity and its viscosity under enzyme activity decreases to 8-10% vs the control 20-27%. Hence, polymer B is not effective for obtaining Solu-NaHA which demonstrates enhanced protection against enzymatic degradation.

### Example 4

### Preparation of Solu-Bovine Serum Albumin (BSA), 2.4%

Preparation of 4.8% solutions of bovine serum albumin (BSA) was carried out by dissolution of 5.0 g of BSA (Merck, K 31587018 320, Albumin from bovine serum, Fraction Y) in 100 ml water at room temperature with mixing on magnet stirrer without heating during 10 min.

NaCl was added to the final concentration of 1.7% (w/w): 1.7 g NaCl to 100 ml 4.8% solution of BSA, and mixed for 5-10 min. 50 ml of Polymer B were placed in a three-necked flask of 150 ml in a water-bath at room temperature (no heating is used for proteins). An equal volume (50 ml) of 4.8% BSA solution was added dropwise to the polymer solution (0.35 ml in 1 minute) with constant mixing by a mechanical glass stirrer utilizing a teflon tip (stirring rate: 300 rpm). The final product, herein designated Solu-BSA, is an opalescent solution, concentration of BSA-2.4%.

The pH, viscosity, size of the particles, and stability under acidic conditions (pH 1.5) were measured. The values obtained were: pH 6.5±0.4, viscosity 11±2 cP. The average particle diameter of the Solu-BSA was 90-120 nm. The stability under acidic conditions was at least for 1.5 hours.

Stability of Solu-BSA under acidic conditions is estimated based on checking for changes in the particle size: absence of change indicates stability. Continuous particle size measurements were made using Malvern light diffraction instrumentation during at least 1.5 hours at temperature 25° C. During this time the disperse system of Solu-BSA remained stable, the average size of particles did not vary.

### Example 5

### Preparation of Solu-Hyaluronidase (Solu-Hd), 0.2%

Preparation of 0.4 solutions of hyaluronidase (Hd) (Sigma, H 3506, Hyaluronidase lyophilized (EC 3.2.1.35) Type I-S, from bovine testes, 608 U/mg solid) was carried out by dissolution of 0.4 g of Hd in 100 ml water at room temperature with mixing on magnet stirrer without heating during 10 min.

NaCl was added to the final concentration of 1.7% (w/w): 1.7 g NaCl to 100 ml 0.4% solution of Hd, and mixed for 5-10 min. 50 ml of Polymer B were placed in a three-necked flask of 150 ml in a water-bath at room temperature. An equal volume (50 ml) of 0.4% Hd solution was added dropwise to the polymer solution (0.35 ml in 1 minute) with constant mixing by a mechanical glass stirrer utilizing a teflon tip (stirring rate: 300 rpm). The final product, herein designated Solu-Hd, is an opalescent solution, concentration of Hd-0.2%.

The pH, viscosity, size of the particles, and stability under acidic conditions (pH 1.5) were measured. The values obtained were: pH 5.0±0.2, viscosity 10±2 cP. The average particle diameter of the Solu-Hd was 150-200 nm. The stability under acidic conditions was at least for 1.5 hours.

Stability is checked by looking changes in particle size. Continuous measurements using Malvern light diffraction instrumentation for at least 1.5 hours at temperature 25° C. were conducted. During this time the disperse system of Solu-Hd remained stable, the average size of particles did not vary.

1. A hydrophilic inclusion complex consisting essentially of nanosized particles of a macromolecule wrapped in an amphiphilic polymer such that non-valent bonds are formed between the macromolecule and the amphiphilic polymer, wherein said amphiphilic polymer is a polysaccharide or a modified polysaccharide.

2. The hydrophilic inclusion complex according to claim 1, wherein said amphiphilic polymer is a polysaccharide selected from the group consisting of starch, chitosan and an alginate.

3. The hydrophilic inclusion complex according to claim 2, wherein said amphiphilic polymer is starch modified to increase its hydrophilicity, or to reduce its branching, or both.

4. The hydrophilic inclusion complex according to claim 3, wherein said amphiphilic polymer is starch modified by a treatment selected from the group consisting of acid hydrolysis, reaction with polyethylene glycol (PEG), reaction with hydrogen peroxide, thermal treatment, and a mixture thereof.

5. The hydrophilic inclusion complex according to claim 4, wherein said amphiphilic polymer is hydrolyzed starch modified by treatment with hydrogen peroxide.

6. The hydrophilic inclusion complex according to claim 1, wherein said macromolecule is a naturally-occurring, synthetic or recombinant polypeptide, protein, polysaccharide or polynucleotide.

7. The hydrophilic inclusion complex according to claim 6, wherein said macromolecule is a naturally-occurring, synthetic or recombinant polypeptide having a molecular weight above 1,000 Da or a protein.

8. The hydrophilic inclusion complex according to claim 7, wherein said polypeptide or protein is a naturally-occurring, synthetic or recombinant hormone, cytokine or chemokine, enzyme, immunoglobulin or monoclonal antibody.

9. The hydrophilic inclusion complex according to claim 8, wherein said naturally-occurring, synthetic or recombinant hormone is insulin, human growth hormone, luteinizing hormone (LH) or human chorionic gonadotropin (hCG).

10. The hydrophilic inclusion complex according to claim 8, wherein said naturally-occurring, synthetic or recombinant cytokine or chemokine is selected from the group consisting of an interferon, an interleukin, granulocyte colony-stimulating factor, granulocyte-macrophage colony-stimulating factor, macropphage colony-stimulating factor, a tumor necrosis factor, a member of the TNF superfamily, a transforming growth factor, erythropoietin and thymopoietin.

11. The hydrophilic inclusion complex according to claim 8, wherein said naturally-occurring, synthetic or recombinant enzyme is trypsin, chymotrypsin, pancreatin, papain, bromelain, fibrinolysin, streptokinase, tissue plasminogen activator, urokinase, hyaluronidase, acid beta-glucocerebrosidase, peroxidase or superoxide dismutase.

12. The hydrophilic inclusion complex according to claim 6, wherein said macromolecule is a polysaccharide selected from the group consisting of hyaluronic acid and its salts, chondroitin sulphate, dermatan sulphate, heparan sulphate, heparin and its derivatives including low molecular weight heparins (LMWH), and lentinan.

13. The hydrophilic inclusion complex according to claim 11, wherein said naturally-occurring, synthetic or recombinant enzyme used is hyaluronidase wrapped in modified corn starch.

14. The hydrophilic inclusion complex according to claim 12, wherein said polysaccharide is hyaluronic acid or its sodium salt wrapped in hydrolyzed starch modified by hydrogen peroxide or in modified corn starch.

15. A hydrophilic dispersion comprising nanoparticles of inclusion complexes consisting essentially of nanosized particles of a macromolecule wrapped in an amphiphilic polymer such that non-valent bonds are formed between the macromolecule and the amphiphilic polymer, wherein said amphiphilic polymer is a polysaccharide or a modified polysaccharide.

16. The hydrophilic dispersion according to claim 15, wherein said amphiphilic polymer is a polysaccharide selected from the group consisting of starch, chitosan and an alginate.

17. The hydrophilic dispersion according to claim 16, wherein said amphiphilic polymer is starch modified to increase its hydrophilicity, or to reduce its branching, or both.

18. The hydrophilic dispersion according to claim 17, wherein said amphiphilic polymer is starch modified by a treatment selected from the group consisting of acid hydrolysis, reaction with polyethylene glycol (PEG), reaction with hydrogen peroxide, thermal treatment, and a mixture thereof.

19. The hydrophilic dispersion according to claim 18, wherein said amphiphilic polymer is modified starch hydrolyzed by treatment with hydrogen peroxide.

20. The hydrophilic dispersion according to claim 15, wherein said macromolecule is a naturally-occurring, synthetic or recombinant polypeptide, protein, polysaccharide or polynucleotide.

21. The hydrophilic dispersion according to claim 20, wherein said macromolecule is a naturally-occurring, synthetic or recombinant polypeptide having a molecular weight above 1,000 Da or a protein.

22. The hydrophilic dispersion according to claim 21, wherein said polypeptide or protein is a naturally-occurring, synthetic or recombinant hormone, cytokine or chemokine, enzyme, immunoglobulin or monoclonal antibody.

23. The hydrophilic dispersion according to claim 22, wherein said naturally-occurring, synthetic or recombinant hormone is insulin, human growth hormone, luteinizing hormone (LH) or human chorionic gonadotropin (hCG).

24. The hydrophilic dispersion according to claim 22, wherein said naturally-occurring, synthetic or recombinant cytokine or chemokine is selected from the group consisting of an interferon, an interleukin, granulocyte colony-stimulating factor, granulocyte-macrophage colony-stimulating factor, macrophage colony-stimulating factor, a tumor necrosis factor, a member of the TNF superfamily, a transforming growth factor, erythropoietin and thymopoietin.

25. The hydrophilic dispersion according to claim 22, wherein said naturally-occurring, synthetic or recombinant enzyme is trypsin, chymotrypsin, pancreatin papain, bromelain, fibrinolysin, streptokinase, tissue plasminogen activator, urokinase, hyaluronidase, acid beta-glucocerebrosidase, peroxidase or superoxide dismutase.

26. The hydrophilic dispersion according to claim 20, wherein said macromolecule is a polysaccharide selected from the group consisting of hyaluronic acid and its salts, chondroitin sulphate, dermatan sulphate, heparan sulphate, heparin and its derivatives including low molecular weight heparins (LMWH), and lentinan.

27. The hydrophilic dispersion according to claim 25, wherein said naturally-occurring, synthetic or recombinant enzyme used is hyaluronidase wrapped in modified corn starch.

28. The hydrophilic dispersion according to claim 26, wherein said polysaccharide is hyaluronic acid or its sodium salt wrapped in hydrolyzed starch modified by hydrogen peroxide or in modified corn starch.

29. A stable composition comprising a dispersion according to claim 15 and a carrier.

30. A stable pharmaceutical composition according to claim 29 comprising said dispersion and a pharmaceutically acceptable carrier.

31. A stable pharmaceutical or cosmetic composition comprising a pharmaceutically acceptable carrier and a hydrophilic dispersion comprising nanoparticles of inclusion complexes consisting essentially of nanosized particles of a macromolecule wrapped in an amphiphilic polymer such that non-valent bonds are formed between the macromolecule and the amphiphilic polymer, wherein said macromolecule is hyaluronic acid or its sodium salt and the amphiphilic polymer is hydrolyzed starch modified by hydrogen peroxide or modified corn starch.

32. A stable pharmaceutical composition comprising a pharmaceutically acceptable carrier and a hydrophilic dispersion comprising nanoparticles of inclusion complexes consisting essentially of nanosized particles of a macromolecule wrapped in an amphiphilic polymer such that non-valent bonds are formed between the macromolecule and the amphiphilic polymer, wherein said macromolecule is hyaluronidase and the amphiphilic polymer is modified corn starch.
